(19) United States
(12) Patent Application Publication
(10) Pub. No.: US 2003/0032602 A1
(43) Pub. Date: Feb. 13, 2003

(54) Methods and compositions for collagen homeostasis

(76) Inventor: Dusan Miljkovic, San Diego, CA (US)

Correspondence Address: RUTAN & TUCKER, LLP, P.O. BOX 1950, COSTA MESA, CA, 92628-1950, US

(21) Appl. No.: 09/927,764

(22) Filed: Aug. 9, 2001

(51) Int. Cl. A61K 31/7024; A61K 9/127

(52) U.S. Cl. 514/23; 424/450; 514/54; 514/561

(57) ABSTRACT

Methods and compositions are directed to a cosmetic preparation comprising a collagenase inhibitor at a concentration effective to reduce a collagenase activity in a skin, wherein the inhibitor comprises boron. Particularly contemplated compositions are topically applied to the skin, and may further comprise a skin penetration enhancer and/or have the collagenase inhibitor formulated in a liposome formulation.

### FIELD OF THE INVENTION

The field of the invention is cosmetic preparations.

### BACKGROUND OF THE INVENTION

Collagen is among the most ubiquitous substances in the body due to its abundant presence in connective tissues. Depending on the type of connective tissue, collagen may be classified into various subclasses, including Type 1 (connective tissue of skin, bone, teeth, tendons, ligaments, fascia, and organ capsules), Type 2 (connective tissue of cartilage), Type 3 (connective tissue of organs), and Type 4/5 (connective tissue of separating layers between epithelial and endothelial cells as well as between skeletal or smooth muscle cells, kidney glomeruli, lens capsules, and Schwann and glial cells of the nervous system).

Unfortunately, the process of ageing in human is frequently accompanied by a decreased production and/or increased degradation of one or more collagen types. While most of the decline in collagen is typically not apparent upon casual obseravtion, diminishing collagen type I and IV quantities often manifest themselves in skin wrinkles, drooping skin, and/or loss of skin elasticity, which many women find particularly undesirable. To counteract the overall loss of collagen in skin, numerous approaches have been tried. For example, collagen can be topically administered to ageing skin, which frequently results in a temporary improvement in skin tone and depth of wrinkles. However, the effects of topically applied collagen are often limited to hydration (i.e., puffing up) of the skin. Alternatively, collagen can be injected into selected areas. Although collagen injection typically has a longer lasting effect, various problems may arise. Among other things, if the injected collagen is not an autotransplant, rejection or allergic reactions are likely to occur. Moreover, even if the collagen is from an autologous source, naturally occurring degradation will eventually degrade the injected collagen as well.

To circumvent at least some of the problems associated with collagen supplementation, formulations can be systemically or topically applied that stimulate the synthesis of collagen. For example, collagen synthesis is known to be stimulated by the systemically administered anabolic steroid stanozolol (see e.g., Falanga et al. in J. Invest. Dermatol. 1998 December; 111(6):1193-7). While steroid-based collagen stimulation may be relatively effective, the use of anabolic steroids is frequently undesirable, especially among women. Moreover, numerous side effects associated with steroids may further discourage a potential user.

Alternatively, systemic application of recombinant growth hormone has been shown to stimulate collagen synthesis (see e.g., “Long-term monitoring of rec-GH treatment by serial determination of serum aminoterminal propeptide of type III procollagen in children and adults with GH deficiency” by Sartorio et al. in Journal Of Endocrinological Investigation (1999), Vol. 22, No. 3.). However, use of growth hormone is relatively expensive. Moreover, depending on the dosages, recombinant growth hormone may exhibit only relatively weak collagen stimulation and may therefore be impracticable to many users.

On the other hand, gamma amino butyrate (GABA) may be used in a topical formulation as described in “Stimulation of human fibroblast collagen synthesis in vitro by gamma-aminobutyric acid” (Biochem Pharmacol 1987; 36(8):1333-1335.). However, GABA is a known neurotransmitter and may pose undesirable systemic side effects upon topical application. Moreover, butyric acid has a relatively offensive odor, thereby potentially limiting its use especially in higher concentrations.

In further known topically applied formulations, vitamin C may be employed to stimulate collagen formulation (see e.g., Fisher, E., McLennan, S V., Tada, H., Heffeman, S., Yue, D K., and Turtle, J R., Interaction of ascorbic acid and glucose on production of collagen and proteogylcan by fibroblasts, Diabetes 1991; 40: 371-375; and Tajima S; Pinnell S R., Ascorbic acid preferentially enhances type I and III collagen gene transcription in human skin fibroblasts., J Dermatol Sci 1996 March; 11(3):250-3). However, relatively high dosages of vitamin C are typically required to significantly improve collagen synthesis, which often tend to provoke an inflammatory response.

Collagen degradation can be inhibited, for example by using TIMP proteins (tissue inhibitors of metalloproteinases). While TIMP proteins are relatively specific towards various types of collagenases, topical use of such proteins is likely to be unsuccessful due to the presence of high concentrations of proteinases on the skin. To reduce potential degradation of polypeptides, certain tripeptides may be employed as collagenase inhibitors (see e.g., U.S. Pat. Nos. 4,687,841 and 4,720,486 to Spilburg, et al.). While smaller peptides are generally more stable towards proteolytic cleavage on the skin, various problems still persist. Among other things, synthetic peptides tend to be relatively potent allergens.

Furthermore, the scientific literature contains references to various further collagenase inhibiting compounds. For example, Clark, et al. (Life Sciences 37: 575-578 (1985)) refer to N[[5-chloro-2-benzothiazolyl)thiophenyl]acetyl]-L-cysteine, said to be a powerful mammalian collagenase inhibitor. Deleaisse, et al. (Biochem Biophys. Res. Comm. 133: 483-490, 1985) also refer to an inhibitor N-[3-N-(benzyloxy-carbonyl)-amino-1-(R)-carboxypropyl]-L-leucy-1-O-methyl-L-tyrosine-N-methylamide. Gray, et al. (Biochem. Biophys. Res. Comm. 101: 1251-1258, 1981) disclose a number of thiol-containing analogues of the collagen cleavage site. Additional thiol-containing peptides are disclosed by Gray, et al. in J. Cell Biochem., 32: 71-77, 1986. Carboxyalkyl peptide analogues are described in Gray, et al. in Federation Proc. 44: 1431, 1985. Miller, et al. and Gray, et al. also disclose thiol-containing peptides in abstracts. [Fed. Proc. 45: 1859 (1986) and FASEB J. 2: A345 (1988), respectively]. Mookhtiar, et al. also discloses phosphonamidate inhibitors of collagenase. (see Biochemistry, 26, 1962 (1987)). However, many of these compounds tend to be problematic in various aspects, particularly including stability, toxicity, and allergenicity.

Thus, although various compositions and methods are known in the art to improve collagen synthesis and/or to reduce collagen degradation, all or almost of them have one or more disadvantages. Therefore, there is still a need for improved methods and compositions to improve collagen levels in skin, and especially in ageing skin.

### BRIEF DESCRIPTION OF THE DRAWING

FIG. 1A is a graph depicting inhibition of collagenase with fluorescence-labeled collagen-I as a substrate using contemplated compounds as inhibitors.

FIG. 1B is a graph depicting inhibition of collagenase with fluorescence-labeled collagen-IV as a substrate using contemplated compounds as inhibitors.

FIG. 1C is a graph depicting inhibition of collagenase with fluorescence-labeled gelatin as a substrate using contemplated compounds as inhibitors.

### SUMMARY OF THE INVENTION

The present invention is directed to methods and compositions for cosmetic preparations comprising a collagenase inhibitor at a concentration effective to reduce a collagenase activity in skin, wherein the collagenase inhibitor comprises boron.

In one aspect of the inventive subject matter, the cosmetic preparation is topically applied to the skin, may comprise a skin penetration enhancer, and in further contemplated formulations the collagenase inhibitor may be formulated in a liposome formulation.

In another aspect of the inventive subject matter, the collagenase inhibitor comprises borate in a complex with at least one ligand, preferably a carbohydrate (e.g., fructose, mannitol, or sorbitol), an amino acid (e.g., serine), or an ascorbate (e.g., ascorbic acid). Especially contemplated reduction of the collagenase activity is at least 20%, more preferably at least 50% for collagen I and collagen IV, wherein the reduction of the collagenase activity reduces the formation of wrinkles in the skin.

In a further aspect of the inventive subject matter, a method of reducing collagenase activity in skin has one step in which a cosmetic preparation is provided that comprises a collagenase inhibitor at a concentration effective to reduce a collagenase activity in a skin, wherein the collagenase inhibitor comprises boron. In a further step, the preparation is delivered to the skin, and preferably topically applied.

Various objects, features, aspects and advantages of the present invention will become more apparent from the following detailed description of preferred embodiments of the invention.

### DETAILED DESCRIPTION

The inventors have surprisingly discovered that certain boron-containing compounds and compositions exhibit collagenase inhibitory effects, and particularly collagenase inhibitory effects in skin. Consequently, the inventors contemplate that a cosmetic preparation may comprise a collagenase inhibitor at a concentration effective to reduce a collagenase activity in a skin, wherein the collagenase inhibitor comprises boron. Particularly contemplated boron-containing compounds have no apparent toxicity and immunogenicity at concentrations effective to inhibit collagenase and are generally regarded safe compounds.

In a particularly preferred aspect of the inventive subject matter, a cosmetic preparation is prepared from commercially available polyethyleneglycolized medium chain triglycerides (e.g., “Labrasol”, “Gatte Fosse Inc.”) (10 g), polyethyleneglycolized triglycerides (e.g., “Gelucir 44”, “Gatte Fosse Inc” (10 g), soybean lecithin granules (Sigma) (5 g) and almond oil (Woodland Nut, Inc.) (20 g). All of these components are well homogenized in a mixer at 40-60 rpm to form fraction A. Separately, fraction B is prepared as a solution from boric acid (0.61 g; 10 mmoles), NaHCO₃ (0.840 g; 10 mmoles) and D-fructose (3.20 g; 20 mmoles) in distilled water (30 ml). Fraction B is then slowly mixed in the component A with a mixer at 40-60 rpm. The so obtained formulation may then further be modified with fragrances, colorants, stabilizers, and/or preservatives.

In alternative aspects of the inventive subject matter, it is contemplated that numerous compositions other than the above described composition for fraction A are also appropriate, and especially contemplated alternative compositions include various creams, ointments, oil-in-water l 5 and water-in-oil emulsions, lotions, milks, gels, mousses, etc. Consequently, the inventors contemplate that all known cosmetic preparations are suitable for use herein, and exemplary compositions and methods of making such compositions are described in “A Formulary of Cosmetic Preparations, Volume 1-Decorative Cosmetics”, by Anthony L. L. Hunting (ISBN: 0-96087522-2), or in “A Formulary of Cosmetic Preparations, Volume 2-Creams, Lotions, and Milks”, by Anthony L. L. Hunting (ISBN: 1-87022809-X).

Furthermore, and especially where contemplated compositions are employed to enhance delivery of suitable collagenase inhibitors to the connective or other tissue to the skin, such formulations may further comprise skin penetration enhancers and/or may include a liposome formulation. There are numerous skin penetration enhancing and liposome formulation known in the art, and it is contemplated that all of such formulations are suitable for use herein.

For example, suitable formulations may include ionic compounds (e.g., ascorbate, calcium thioglycolate, cetyl trimethyl ammonium bromide, ionic surfactants, 5-methoxysalicylate, etc.), dimethyl sulfoxide and related compounds (e.g., cyclic sulfoxides, decylmethyl sulfoxide, etc.), azone and related compounds (e.g., 1-dodecyl azacycloheptan-2-one, N-Dodecyl-2-pyrrolidone, azacycloalkane derivatives, 1-geranylazacycloheptan-2-one, etc.). Further contemplated skin penetration enhancer include solvents (e.g., alkanols, esp. ethanol, dimethyl formamide, polyoxyethylene sorbitan monoesters, propylene glycol, etc.), or fatty alcohols, fatty acids, and related structures (e.g., aliphatic and lauryl alcohols, dodecyl N,N-dimethylamino acetate, ethyl acetate, alkanoic acids and oleic acids, isopropyl myristate, etc.). Still further contemplated formulations include enzymes (e.g., papain), amines and amides (e.g., N,N-Diethyl-m-toluamide), complexing agents (e.g., Brij, Pluronic, etc), and N-methyl pyrrolidone and related compounds (e.g., 1,3-Dimethyl-2-imidazolikinone or 2-Pyrrolidone).

Preparation of exemplary contemplated formulations is described e.g., in “Percutaneous Penetration Enhancers” by Eric W. Smith and Howard I. Maibach (CRC Press; ISBN: 0849326052), or in “Pharmaceutical Skin Penetration Enhancement” by Kenneth A. Walters, Jonathan Hadgraft (Marcel Dekker; ISBN: 0824790170), or in “Topical Drug Bioavailability, Bioequivalence, and Penetration” by Vinod P. Shah and Howard I. Maibach (Plenum Pub Corp; ISBN: 0306443678). Preparation of liposome formulations can be found in “Liposome Technology: Liposome Preparation and Related Techniques” by Gregory Gregoriadis (CRC Press; ISBN: 0849367077), or in “Liposome Methods and Protocols (Methods in Molecular Biology)” by Subhash C. Basu and Manju Basu (Humana Press; ISBN: 0896038459).

With respect to fraction B is it contemplated that numerous alternative preparations are also appropriate, and it should be appreciated that the chemical nature of the collagenase inhibitor may vary significantly so long as the inhibitor still comprises a boron atom. For example, where the use of boric acid is less desirable, corresponding salts (Na⁺ or K⁺) may be employed. Alternatively, borax (Na₂B₄O₅(OH)₄*8H₂O), kernite (Na₂B₄O₅(OH)₄*2H₂O), or other naturally occurring forms of boron may be suitable. In a particularly preferred aspect of the inventive subject matter, boron is present as a borate and is complexed by at least one ligand, and especially preferred ligands include carbohydrates (e.g., fructose, glucose, mannitol, or sorbitol), amino acids (e.g., serine), and ascorbates (e.g., vitamin C). There are numerous boron-complexes known in the art and particularly preferred complexes are described in U.S. Pat. Nos. 5,962,049, 5,985,842, and 6,080,425, all of which are incorporated by reference herein.

Furthermore, the amount of suitable collagenase inhibitors need not be limited to a specific amount, and it should be appreciated that all amounts are suitable that will produce a collagenase inhibition in skin when the formulation is applied to the skin. Thus, appropriate amounts of contemplated inhibitors will typically be between 0.1 wt % and 5 wt %. However, where appropriate, amounts between 0.1 wt % and 0.01 wt % and even less are also contemplated. Similarly, and especially where relatively high concentrations of contemplated inhibitors are desired, appropriate amounts of contemplated inhibitors may also be between 5 wt % and 25 wt % (and even more).

Depending on the particular inhibitor and ligand, it should further be appreciated that the solvent may vary accordingly. Thus, suitable solvents may include aqueous and non-aqueous solvents, and particularly include water, ethanol, dimethylformamide, dimethylsulfoxide, glycerol, and all chemically reasonable combinations thereof. Moreover, it should further be appreciated that contemplated preparations need not be limited to compositions and methods in which a fraction A is combined with a fraction B, and it is contemplated that in alternative compositions and methods suitable collagenase inhibitors may be included in a single fraction, or more than one fractions.

Similarly, various protocols for application of contemplated compounds are contemplated, and a particular protocol will typically depend on the particular formulation. However, it is generally preferred that contemplated formulations are topically applied to the skin of a person at least once a week, more preferably at least once daily in an amount that typically will not exceed absorption of the formulation into the skin. For example, particularly suitable applications include facial creams, lipsticks, sunscreens, and body lotions, all of which maybe applied to reduce degradation of collagen, and thereby to reduce appearance of wrinkles in the skin.

Consequently, method of reducing collagenase activity in skin may comprise one step in which a cosmetic preparation is provided that comprises a collagenase inhibitor at a concentration effective to reduce a collagenase activity in a skin, wherein the collagenase inhibitor comprises boron. In a further step, the preparation is applied to the skin (e.g., massaged into the skin).

### EXAMPLES

The following examples have been provided to illustrate various aspects of the inventive subject matter. More particularly, all examples employed commercially available collagenase from Clostridium histolyticum (EC 3.4.24.3; Fluka Chemie AG, Switzerland) and commercially available collagen-I and collagen-IV (Molecular Probes, OR, USA).

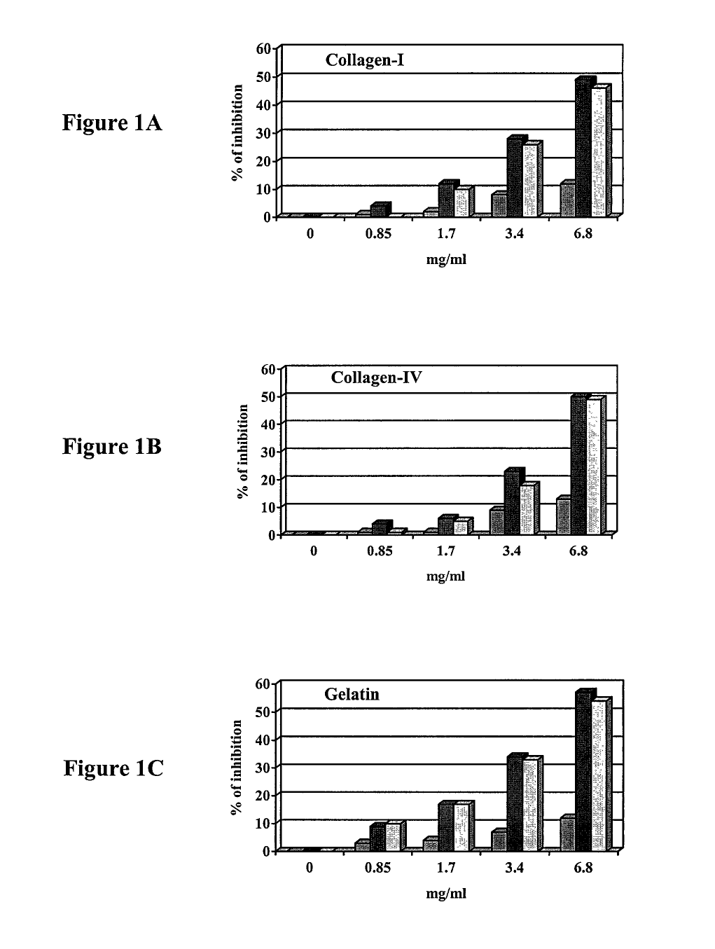

Inhibition of collagenase from Clostridium histolyticum was determined using fluorophor labeled Gelatin-FL, Collagen I-FL and Collagen IV-FL as substrate. Negative control was Mg ascorbyl-2-phosphate (MAP), and exemplary boron-containing compounds were AB(F-D) and AB (V-E), which represented two different batches of potassium ascorbato-borates. The assay was performed under standard conditions with incubation and of substrate for 60 min at 37° C. and fluorescence was measured in a standard fluorometer. The results of the inhibition experiments are depicted in FIGS. 1A-1C. With respect to the inhibitors, the first column represents the negative control MAP, while the second and third columns represent exemplary boron-containing compounds AB(F-D) (potassium ascorbato-borate) and AB(V-E) (potassium ascorbato-borate). As can be clearly seen, exemplary boron-containing compounds are specific inhibitory compounds for collagenase with respect to various collagen types (I and IV and denatured). Moreover, additional experiments (data not shown) confirmed the inhibitory character of numerous other boron-containing compounds, and especially boron-containing compounds in which borate is complexed by one or more ligands.

While the here described experiments are in vitro experiments, it is specifically contemplated that the boron-containing compounds presented herein will also be available to collagenases within the skin of a mammal (and particularly human), especially when contemplated compounds are formulated in a skin penetration enhancing or liposome containing formulation (see e.g., “Permeation enhancers for transdermal drug delivery” by Sinha and Kaur, Drug Dev Ind Pharm 2000 November; 26(11):1131-40). Furthermore, it is contemplated that even without use of penetration enhancers contemplated inhibitors exhibit a sufficiently small molecular weight to pass through at least some layers of skin (see e.g., “The 500 Dalton rule for the skin penetration of chemical compounds and drugs” by Bos and Meinardi; Exp Dermatol 2000 June; 9(3):165-9). Still further, tests on human volunteers (data not shown) clearly indicated reduction of skin wrinkles and increase of skin tone in various areas, including the face and upper side of the hand.

Thus, specific embodiments and applications of methods and compositions for collagen homeostasis have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A cosmetic preparation comprising a collagenase inhibitor at a concentration effective to reduce a collagenase activity in a skin, wherein the collagenase inhibitor comprises boron.

2. The cosmetic preparation of claim 1 wherein the preparation is topically applied to the skin.

3. The cosmetic preparation of claim 2 further comprising a skin penetration enhancer.

4. The cosmetic preparation of claim 2 wherein the collagenase inhibitor is formulated in a liposome formulation.

5. The cosmetic preparation of claim 1 wherein the collagenase inhibitor comprises borate in a complex with at least one ligand.

6. The cosmetic preparation of claim 5 wherein the ligand is selected from the group consisting of a carbohydrate, an amino acid, and an ascorbate.

7. The cosmetic preparation of claim 6 wherein the carbohydrate is selected from the group consisting of a fructose, a mannitol, and a sorbitol.

8. The cosmetic preparation of claim 6 wherein the amino acid is serine.

9. The cosmetic preparation of claim 1 wherein the collagenase activity comprises hydrolysis of collagen I and collagen IV.

10. The cosmetic preparation of claim 9 wherein the reduction of the collagenase activity is at least 20% for collagen I and collagen IV.

11. The cosmetic preparation of claim 9 wherein the reduction of the collagenase activity is at least 50% for collagen I and collagen IV.

12. The cosmetic preparation of claim 1 wherein the reduction of the collagenase activity reduces appearance of wrinkles in the skin.

13. A method of reducing collagenase activity in skin, comprising:
providing a cosmetic preparation comprising a collagenase inhibitor at a concentration effective to reduce a collagenase activity in a skin, wherein the collagenase inhibitor comprises boron; and
applying the preparation to the skin.

14. The method of claim 13 wherein the step of applying the preparation comprises massaging the preparation into the skin.

15. The method of claim 13 wherein the cosmetic preparation cosmetic comprises a skin preparation enhancer.

16. The method of claim 13 wherein the collagenase inhibitor is formulated in a liposome formulation.

17. The method of claim 13 wherein the collagenase inhibitor comprises borate in a complex with at least one ligand.

18. The method of claim 13 wherein the ligand is selected from the group consisting of a carbohydrate, an amino acid, and an ascorbate.

19. The method of claim 13 wherein the collagenase activity comprises hydrolysis of collagen I and collagen IV.

20. The method of claim 13 wherein the reduction of the collagenase activity reduces the formation of wrinkles in the skin.
